(19) United States
(12) Patent Application Publication
(10) Pub. No.: US 2024/0108866 A1
(43) Pub. Date: Apr. 4, 2024

(54) Preventing Collapse or Narrowing of a Through Lumen of a Catheter Shaft in a Balloon Guide Catheter Hub During Aggressive Inflation

(71) Applicant: Neuravi Limited, Galway (IE)

(72) Inventor: Richard CONLON, Galway (IE)

(21) Appl. No.: 17/936,828

(22) Filed: Sep. 29, 2022

(51) Int. Cl. A61M 25/10

(52) U.S. Cl. CPC A61M 25/10184; A61M 25/1029; A61M 25/1034; A61M 2207/10

(57) ABSTRACT

A balloon guide catheter including an injection molded balloon guide catheter hub including a through lumen and a balloon inflation lumen converging therewith defining a region of convergence. The injection molded balloon guide catheter hub includes a preventative structure withstanding, avoiding or counterbalancing pressure peaks in the injection molded balloon guide catheter hub during aggressive inflation of the balloon.

### FIELD OF THE INVENTION

The present invention relates to an injection molded balloon guide catheter hub used in intravascular treatment procedures (e.g., during a thrombectomy procedure to remove a clot). In particular, the present invention is directed to an injection molded balloon guide catheter hub with a preventative structure to prevent collapse or narrowing of a through lumen of the of the catheter shaft when assembled therein during aggressive inflation of the balloon. The preventative structure may be in the form of: a reinforcing structure in the clearance space between the through lumen of the injection molded balloon guide catheter hub and the catheter shaft assembled therein to prevent collapse or narrowing of the through lumen of the catheter shaft when subject to spikes in pressure; restricting the rate of flow of inflation media through the balloon inflation lumen of the molded balloon guide catheter hub to avoid spikes in pressure generated by aggressive inflation of the balloon; and/or increasing the internal volume of the balloon inflation lumen of the balloon guide catheter hub to counterbalance a spike in pressure produced during aggressive inflation of the balloon.

### DESCRIPTION OF RELATED ART

Balloon guide catheters are commonly used during intravascular treatment procedures. One commonly performed treatment is a thrombectomy procedure wherein the inflated balloon is used to temporarily arrest blood flow to facilitate the capture and removal of an occlusion, blockage, thrombus, or clot from an artery using a mechanical retrieval device (e.g., stent retriever) and/or through aspiration. FIG. 1A is an assembly including a conventional injection molded balloon guide catheter hub 10 having a balloon inflation lumen luer 25 at an entrance of a balloon inflation lumen 30 and a through lumen luer 15 at an entrance of a through lumen 20 (FIG. 1C) for connecting thereto an accessory (e.g., a hemostasis valve). Assemblable in the through lumen 20 of the balloon guide catheter hub 10 is a catheter shaft 300 having a through lumen 325 and an eccentrically arranged balloon inflation lumen 335. When properly assembled together a proximal inflation entrance port 335a of the balloon inflation lumen 335 of the catheter shaft 300 is in fluid communication with the balloon inflation lumen 30 of the balloon guide catheter hub 10, while a distal inflation exit port 335b of the balloon inflation lumen 335 of the catheter shaft 300 in fluid communication with a compliant balloon 50. FIG. 1B is a partial perspective view from a proximal end 301 of the balloon guide catheter shaft 300 of FIG. 1A (without the injection molded balloon guide catheter hub 10) depicting the proximal inflation entrance port 335a of the balloon inflation lumen 335 arranged eccentrically of the through lumen 325. Within the injection molded balloon guide catheter hub 10, the balloon inflation lumen 30 converges with the through lumen 20 in a Y-shape configuration denoting a region of convergence. Distally of the region of convergence between the two lumen 20, 30 of the injection molded balloon guide catheter hub 10, the balloon inflation lumen 335 and the through lumen 325 of the catheter shaft 300 inserted therein run continuously parallel with each other (i.e., without cross-link or fluid communication).

The catheter shaft 300 is assembled in the through lumen 20 of the injection molded balloon guide catheter hub 10. An adhesive is then injected through adhesive ports defined in the hub's through lumen 20 on each side of a region or interface of convergence of the lumens 20, 30. In the space between the catheter shaft 300 and the hub's through lumen 20 the injected adhesive creates two radial adhesive bonds longitudinally/axially separated from each other forming therebetween a gap (i.e., spanning the region of convergence/interface of the lumens 20, 30) that remains free or devoid of any adhesive.

During treatment, the catheter shaft 300 with a balloon 50 secured about its outer surface proximate the distal section is advanced through the vasculature to a location on the distal side of the target clot while the balloon is in a deflated state. Next, inflation media (e.g., a solution or mixture of contrast agent and saline solution) injected via a syringe connected to the balloon inflation lumen luer 25 passes through the balloon inflation lumen 30 of the injection molded balloon guide catheter hub 10 and via the proximal inflation entrance port 335a into the balloon inflation lumen 335 of the catheter shaft 300 inflating the balloon 50 thereby arresting blood flow distally of the clot. Depending on such factors as the viscosity of the contrast agent in the inflation media solution and/or the size of the syringe (e.g., approximately 1 ml), undesirable peaks or spikes in pressure may occur within the balloon guide catheter hub 10 when the rate of inflation media dispensed from the syringe exceeds the maximum rate of flow achievable through the balloon inflation lumen 30, hereby defined as “aggressive inflation.” These peaks/spikes in pressure in the injection molded balloon guide catheter hub may disadvantageously result in possible collapse or narrowing of the through lumen 325 of the catheter shaft 300 in an area of the region of convergence “S” in which the two lumens 20, 30 converge. In the event of complete collapse or narrowing of the through lumen 20 of the catheter shaft 300, passage of an auxiliary device (e.g., intermediate catheter, microcatheter or stent retriever) therethrough would be hampered or prevented altogether.

It is desirable to develop an improved injection molded balloon guide catheter hub preventing collapse or narrowing of the through lumen of the catheter shaft assembled therein during aggressive inflation of the balloon.

### SUMMARY OF THE INVENTION

An aspect of the present invention is directed to an injection molded balloon guide catheter hub that during aggressive inflation of the balloon prevents collapse or narrowing of the through lumen of the catheter shaft assembled therein by: forming a reinforcing structure disposed in the clearance space between the catheter shaft and the through lumen of the injection molded balloon guide catheter hub so as to withstand spikes in pressure; restricting the rate of flow of inflation media through the balloon inflation lumen of the injection molded balloon guide catheter hub to avoid spikes in pressure; and/or increasing the internal volume of the balloon inflation lumen within the injection molded balloon guide catheter hub to counterbalance or compensate for the occurrence of spikes in pressure.

Another aspect of the invention is directed to a balloon guide catheter including an injection molded balloon guide catheter hub including a through lumen and a balloon inflation lumen converging therewith defining a region of convergence. The injection molded balloon guide catheter hub includes a preventative structure withstanding, avoiding or counterbalancing peaks or spikes in pressure in the injection molded balloon guide catheter hub during aggressive inflation of the balloon.

Still yet another aspect of the invention relates to a method of manufacture of a balloon guide catheter hub. Two halves of a balloon guide catheter hub mold are assembled, aligned and secured together to define therein a first internal cavity for forming a through lumen of an injection molded balloon guide catheter hub and a second internal cavity for forming a balloon inflation lumen of the injection molded balloon guide catheter hub; the first internal cavity converging with the second internal cavity along a region of convergence. A multi-component hub mold core pin comprising three components including a first component, a second component, and a third component are introduced into the assembled two halves of the balloon guide catheter hub mold. The first component has a plurality of fins extending in a longitudinal direction and radially outward from an outer surface thereof. Each of the first and second components are inserted into the first internal cavity of the assembled two halves of the balloon guide catheter hub mold in proximal and distal directions, respectively. The third component is inserted in a distal direction into the second internal cavity of the assembled two halves of the balloon guide catheter hub mold. A flowable material is injected into the assembled two halves of the balloon guide catheter mold filling: (i) the first internal cavity surrounding the first and second components of the multi-component hub mold core pin disposed therein forming the through lumen of the injection molded balloon guide catheter hub; and (ii) the second internal cavity surrounding the third component of the hub mold core pin disposed therein forming the balloon inflation lumen of the injection molded balloon guide catheter hub. A tool is then pressed radially inward through the injected flowable material in the first internal cavity forming the through lumen within the injection molded balloon guide catheter hub to define a plurality of adhesive ports therein, at least one adhesive port formed on each of a proximal side and a distal side of the region of convergence of the hub's through and balloon inflation lumens. The injected flowable material in the balloon guide catheter hub mold is then cooled. Next, each of three components of the multi-component hub mold core pin are independently withdrawn from the assembled two halves of the balloon guide catheter hub mold. The assembled two halves of the balloon guide catheter hub mold are opened, revealing the injection molded balloon guide catheter hub having along an axial section of the through lumen spanning the region of convergence in which the balloon inflation lumen and the through lumen of the injection molded balloon guide catheter hub converge a plurality of longitudinal channels formed by the plurality of fins of the first component of the multi-component hub mold core pin during the injection of the flowable material into the assembled two halves of the balloon guide catheter hub mold.

While still a further aspect of the invention is directed to a method of operating a balloon guide catheter that includes: a catheter shaft having a through lumen extending from a proximal end to an opposite distal end and an eccentrically arranged balloon inflation lumen; a balloon is secured about the catheter shaft; and an injection molded balloon guide catheter hub including a through lumen for receiving therein the proximal end of the catheter shaft and a balloon inflation lumen converging therewith. The method of operation being that during aggressive inflation of the balloon, collapse or narrowing of the through lumen of the catheter shaft in the region of convergence is prevented via a mechanical preventative structure disposed in the balloon inflation lumen within the injection molded balloon guide catheter hub.

### BRIEF DESCRIPTION OF THE DRAWING

The foregoing and other features of the present invention will be more readily apparent from the following detailed description and drawings illustrative of the invention wherein like reference numbers refer to similar elements throughout the several views and in which:

FIG. 1A illustrates a side view of a Prior Art injection molded balloon guide catheter hub and catheter shaft assembly;

FIG. 1B is a perspective view of a proximal section of the catheter shaft of FIG. 1A depicting the through lumen and the eccentrically arranged balloon inflation lumen;

FIG. 1C is a longitudinal cross-sectional view of the injection molded balloon guide catheter hub of FIG. 1A (without the catheter shaft) depicting the through lumen and balloon inflation lumen;

FIG. 1D is an enlarged view of the region of convergence “S” of the through lumen and balloon inflation lumen of the injection molded balloon guide catheter hub of FIG. 1C;

FIG. 2A is an exploded perspective view of an exemplary three-component hub mold core pin in accordance with the present invention;

FIG. 2B is an enlarged distal end view of the three-component hub mold core pin of FIG. 2A with the components assembled together;

FIG. 2C is an enlarged radial cross-sectional view of the three-component hub mold core pin of FIG. 2A along lines II(C)-II(C) of the first component;

FIG. 2D illustrates each of the individual components comprising the three-component hub mold core pin of FIG. 2A assembled in respective internal cavities of the hub mold one half of which is depicted in FIG. 9;

FIG. 2E is a proximal end perspective view of the present inventive formed injection molded balloon guide catheter hub following withdraw of each of the components of the three-component hub mold core pin and removal of the hub mold;

FIG. 2F is a distal end perspective view of the present inventive formed injection molded balloon guide catheter hub following withdraw independently of each of the components of the three-component hub mold core pin and removal of the hub mold;

FIG. 3A is a perspective internal view of a multi-lumen catheter shaft assembled within the through lumen of the present inventive formed injection molded balloon guide catheter hub of FIG. 2E illustrating the radial and longitudinal bonds formed in the clearance space therebetween by a biocompatible adhesive injected via adhesive ports;

FIG. 3B is a radial cross-sectional view through the assembly of FIG. 3A along lines III(B)-III(B) (i.e., through the distal radial bond);

FIG. 3C is a radial cross-sectional view through the assembly of FIG. 3A along lines III(C)-III(C) (i.e., through the proximal radial bond);

FIG. 3D is a radial cross-sectional view through the assembly of FIG. 3A along lines III(D)-III(D) (i.e., through the internal axial/longitudinal bonds);

FIG. 4A is a perspective view of a present inventive flow restrictor for reducing or preventing the occurrence of pressure spikes/peaks in the balloon inflation lumen of the injection molded balloon guide catheter hub;

FIG. 4B is a side view of the flow restrictor of FIG. 4A secured via adhesive in the balloon inflation lumen of the injection molded balloon guide catheter hub, wherein the narrow inner diameter of the channel of the flow restrictor through which the inflation media flows, reduces or prevents the occurrence of pressure spikes/peaks in the balloon inflation lumen of the injection molded balloon guide catheter hub;

FIG. 5A is a side view of an alternative present inventive self-transitioning flow restrictor transitionable between an engaged state (during inflation of the balloon) and a disengaged state (during deflation of the balloon);

FIG. 5B depicts a partial view of the self-transitioning flow restrictor of FIG. 5A disposed in the balloon inflation lumen of the injection molded balloon guide catheter hub, wherein the self-transitioning flow restrictor is depicted in an engaged (“ON”) state during inflation of the balloon with the flow of inflation media restricted exclusively through the restrictor needle;

FIG. 5C depicts a partial view of the self-transitioning flow restrictor of FIG. 5A disposed in the balloon inflation lumen of the injection molded balloon guide catheter hub, wherein the self-transitioning flow restrictor is depicted in a disengaged (“OFF”) state during deflation of the balloon with the flow of inflation media permitted through the internal cavity of the body of the flow restrictor, around the inverted (conical shape) flexible seal reduced in diameter, and through the restrictor needle;

FIG. 6 is a side view of an elastomeric sleeve secured via adhesive about a plurality of slits defined in the wall of the balloon inflation lumen of the injection molded balloon guide catheter hub; the elastomeric sleeve expanding/swelling thereby increasing the internal volume within the hub's balloon inflation lumen and compensating/counterbalancing for pressure spikes that exceed a predetermined threshold during aggressive inflation of the balloon;

FIG. 7A is a side view of elastomeric patch (e.g., blister) secured about an opening defined in the outer surface of the balloon inflation lumen of the injection molded balloon guide catheter hub via a flange; wherein the elastomeric patch (e.g., blister) expands/swells thereby increasing the internal volume within the hub's balloon inflation lumen and compensates/counterbalances spikes/peaks in pressure that exceed a predetermined threshold during aggressive inflation of the balloon; the elastomeric patch is depicted in an expanded/swelled state;

FIG. 7B is an enlarged view of the elastomeric patch (e.g., blister) of FIG. 7A;

FIG. 8 is a side view of an injection molded balloon guide catheter hub having a multi-component balloon inflation lumen of which one component is an elastomeric tube expandable radially and/or longitudinally; the individual components comprising the hub's multi-component balloon inflation lumen being secured in proper alignment both longitudinally and radially via a rail-and-guide system; and

FIG. 9 is a perspective internal view of a one half of an exemplary mold cavity for producing an injection molded multi-lumen balloon guide catheter hub, prior to insertion of the 3-component hub mold core pin of FIG. 2A and injection of the meltable material therebetween.

### DETAILED DESCRIPTION OF THE INVENTION

In the description, the terms “distal” or “proximal” are used in the following description with respect to a position or direction relative to the treating physician or medical interventionalist. “Distal” or “distally” are a position distant from or in a direction away from the physician or interventionalist. “Proximal” or “proximally” or “proximate” are a position near or in a direction toward the physician or medical interventionist. The terms “occlusion”, “clot” or “blockage” are used interchangeably.

Depending on such factors as the viscosity of the contrast agent in the inflation media solution and/or the size of the syringe (e.g., approximately 1 ml), during inflation of the balloon when the rate of inflation media dispensed from the syringe exceeds the maximum rate of flow achievable through the hub's balloon inflation lumen (herein expressly defined as “aggressive inflation”) peaks or spikes in pressure may be produced in the injection molded balloon guide catheter hub. In accordance with the present invention, undesirable collapse or narrowing along a section of the through lumen of the catheter shaft coinciding with the region of convergence of the through and balloon inflation lumens of the injection molded balloon guide catheter hub resulting from peaks or spikes in pressure of the inflation media during aggressive inflation of the balloon is avoided by employing one or more preventative structures described herein.

In accordance with the present invention, the preventative structure may be: (i) a reinforced section disposed within the clearance space between the catheter shaft and through lumen of the injection molded balloon guide catheter hub spanning the region of convergence between the through and balloon inflation lumens of the injection molded balloon guide catheter hub, such reinforced section is able to withstand peaks or spikes in pressure; (ii) a flow restrictor associated with the balloon inflation lumen of the injection molded balloon guide catheter hub restricting the rate of flow of inflation media thereby avoiding peaks or spikes in pressure; and/or (iii) a mechanical device associated with the balloon inflation lumen of the injection molded balloon guide catheter hub increasing the internal volume of the hub's balloon inflation lumen so as to compensate/counterbalance peaks or spikes in pressure.

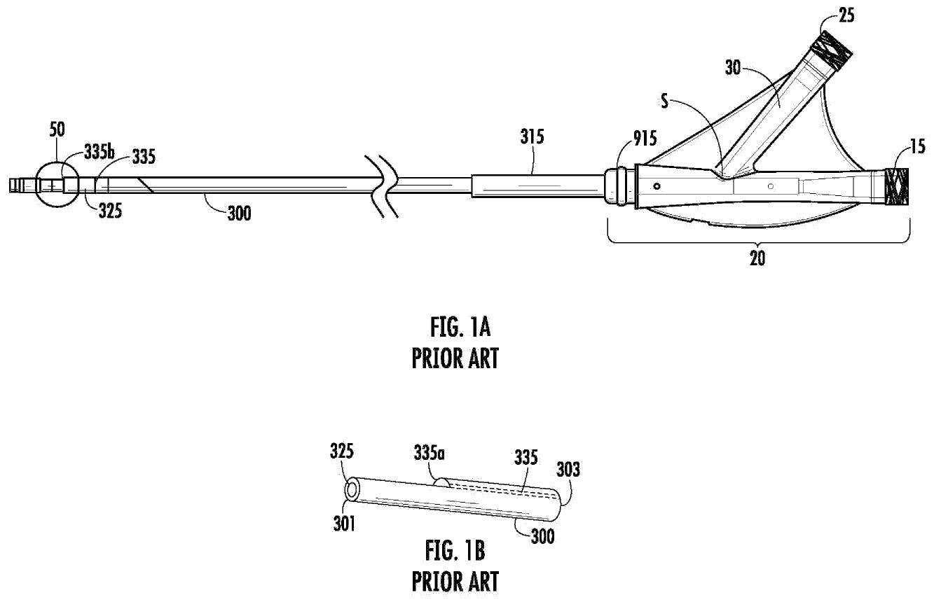

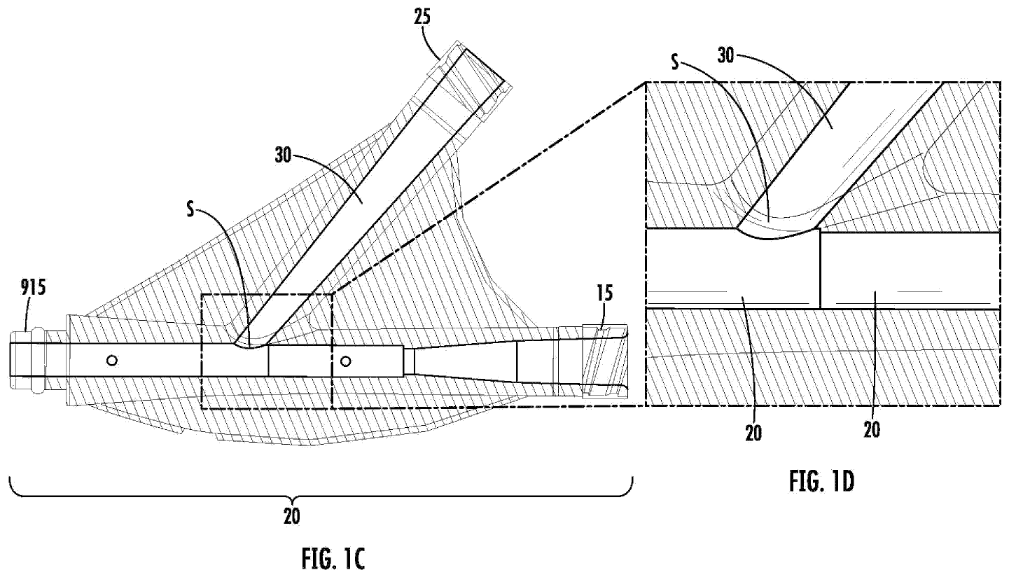

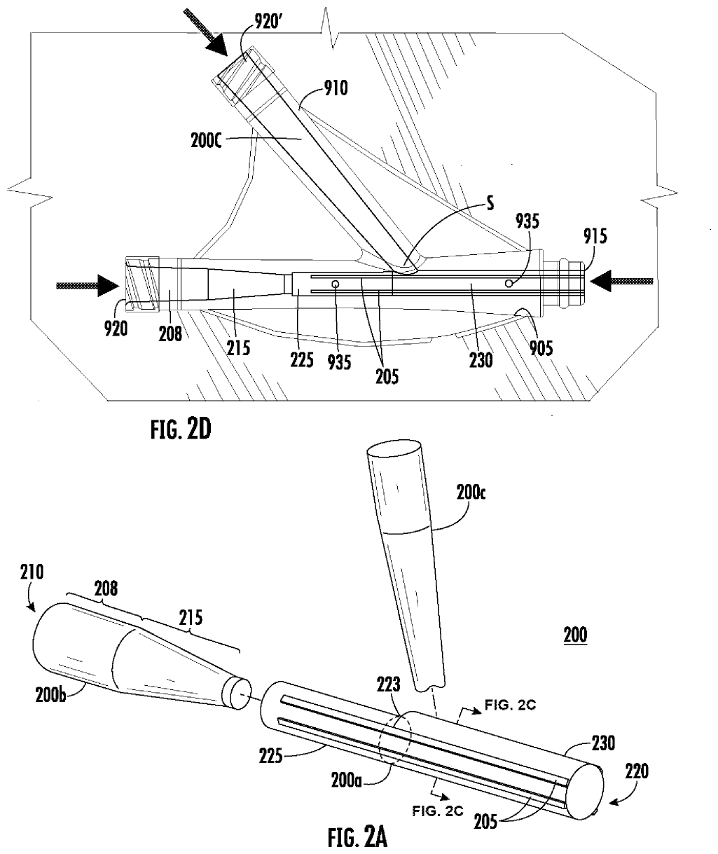

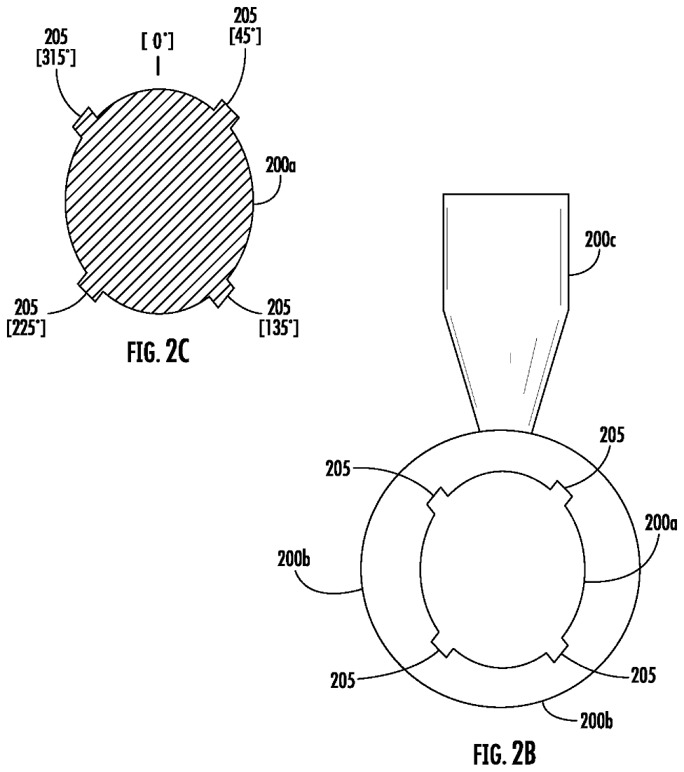

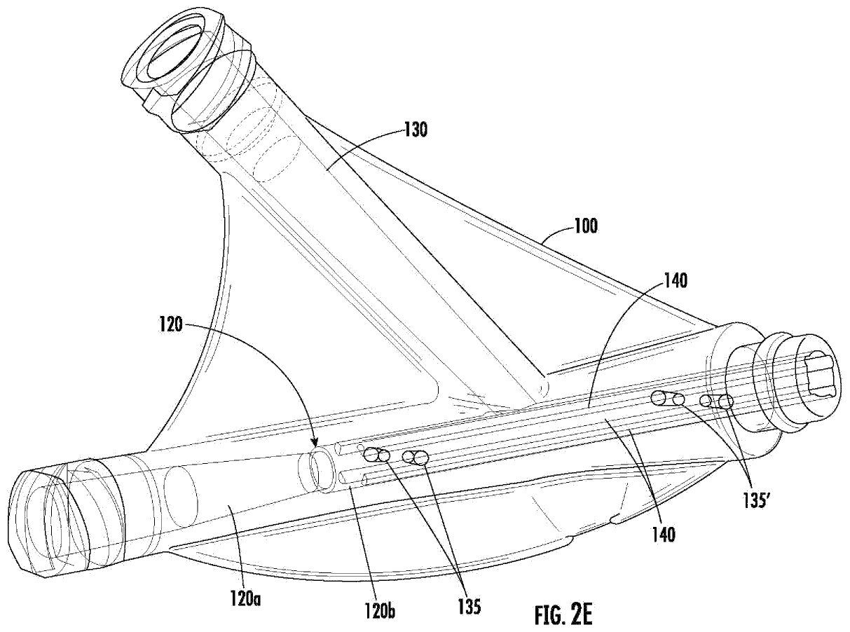

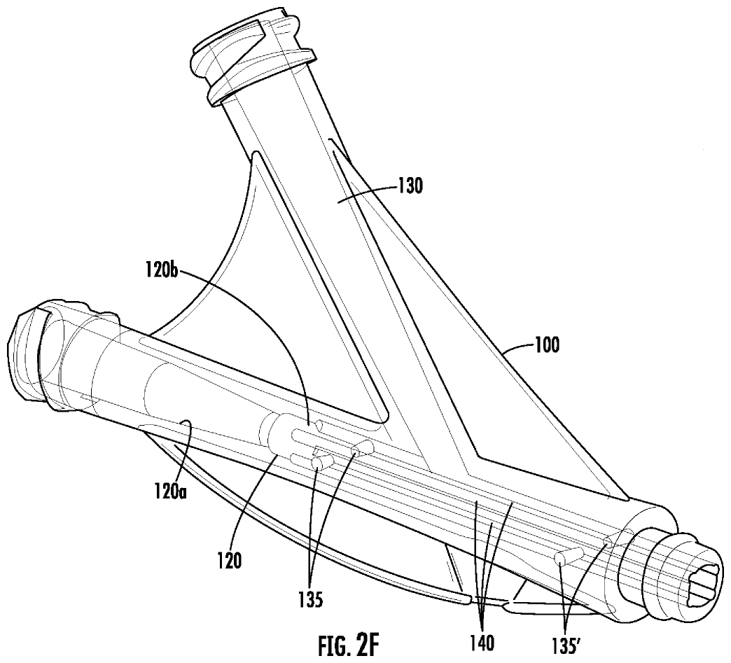

The multi-lumen injection molded balloon guide catheter hub includes: a through lumen extending longitudinally therein from a proximal end to an opposite distal end; and a side balloon inflation lumen in fluid communication with a balloon inflation lumen of a catheter shaft when assembled in the through lumen of the hub. A region of convergence is defined in the injection molded balloon guide catheter hub at a transition or interface where the hub's balloon inflation lumen and the hub's through lumen converge with one another. When the catheter shaft is properly assembled in the distal end of the through lumen of the injection molded balloon guide catheter hub, the proximal entrance port 335a of the inflation lumen 335 of the catheter shaft 300 is just distal of the region of convergence “S” of the hub's through lumen and hub's balloon inflation lumen (FIG. 2D). Proper assembly of the catheter shaft 300 in the through lumen of the injection molded balloon guide catheter hub 10 is indicated by aligning with one another of the “*” denoted in FIGS. 1B & 2D.

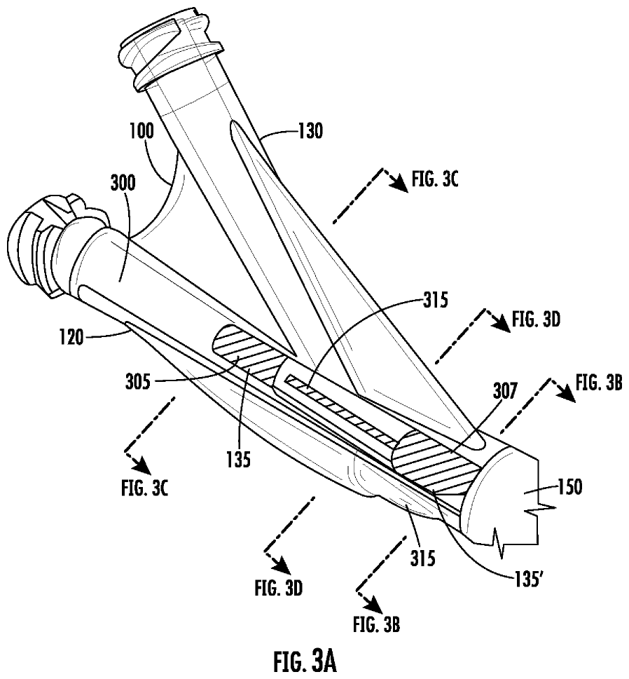

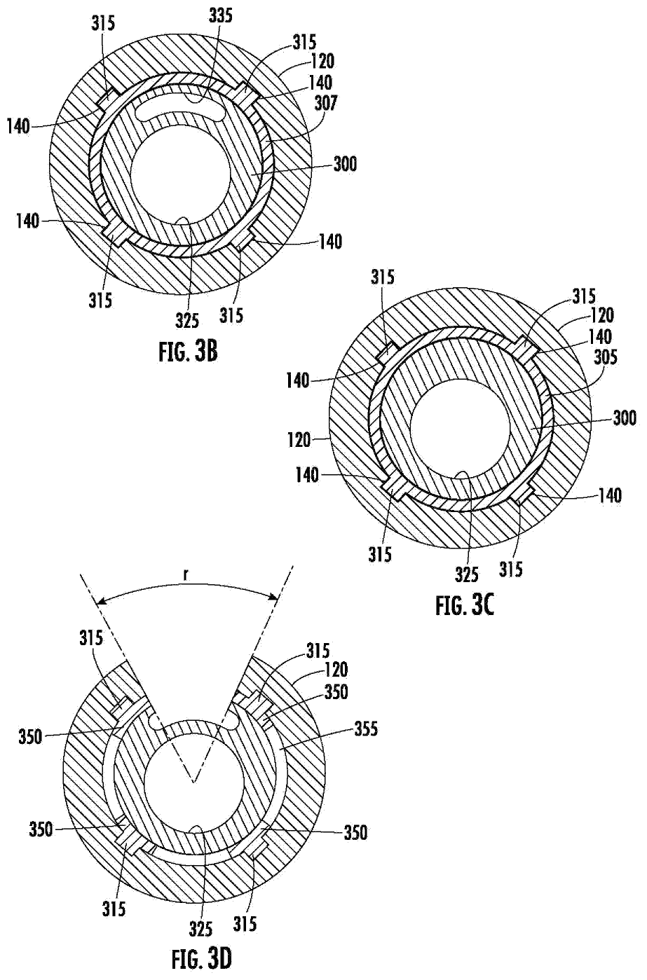

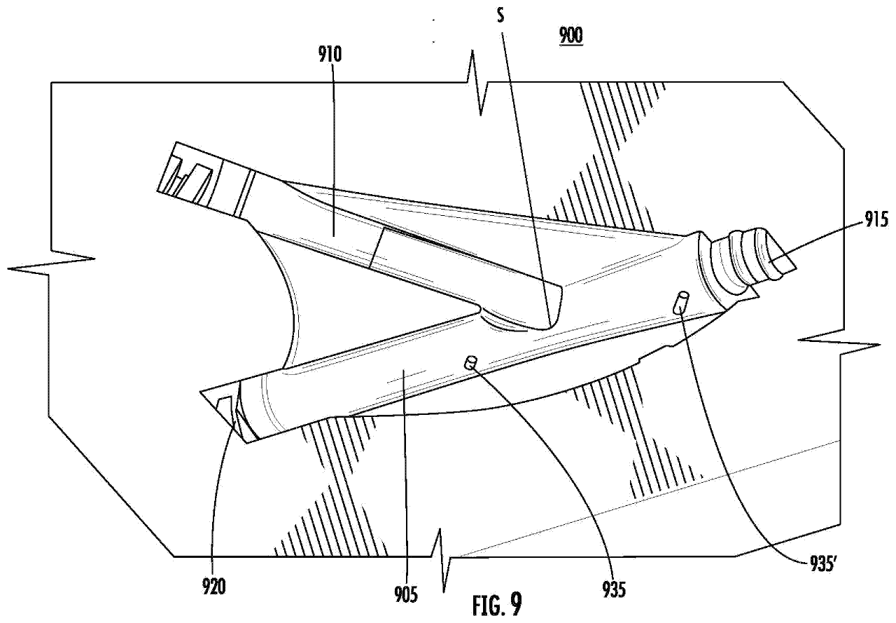

At the time of manufacture during the injection molding process of the multi-lumen balloon guide catheter hub, a portion of the hub's through lumen (e.g., main or central lumen) may be structurally modified (reinforced) in accordance with the present invention to prevent or minimize collapse of the through lumen of the catheter shaft assembled therein during aggressive inflation of the balloon. Specifically, the hub's through lumen is structurally modified spanning a region of convergence with the hub's balloon inflation lumen. During the injection molding process for manufacture of the balloon guide catheter hub a supplemental structure is created within the hub's through lumen, spanning in a longitudinal/axial direction, without blocking, restricting, or interfering with the region of convergence (i.e., fluid communication) with the hub's balloon inflation lumen. A hub mold 900 (one half of which is depicted in FIG. 9, the other half not shown being identical thereto) is used during the injection molding process for manufacture of the injection molded balloon guide catheter hub 100 including the through lumen 120 and the balloon inflation lumen 130 converging (i.e., in fluid communication) therewith in a “Y” shape configuration. The injection molded multi-lumen balloon guide catheter hub is formed using the hub mold 900 (one half of which is depicted in FIG. 9) comprising two internal cavities, namely, a first internal cavity 905 forming the hub's through lumen 120 and a second internal cavity 910 forming the hub's balloon inflation lumen 130. First internal cavity 905 forms a through lumen luer 920 at its proximal end and a protruding lug 915 at the opposite distal end, while second internal cavity 910 forms a balloon inflation lumen luer 920′ at his proximal end. A strain relief 150 (shown in FIG. 3) is positioned about the protruding lug 915 and retained in place by interaction with a radial bump therein. These two internal cavities 905, 910 of the hub mold 900 are in fluid communication along a region of convergence “S” with one another. However, once the catheter shaft 300 is assembled in the hub's through lumen the through lumen and balloon inflation lumen of the injection molded balloon guide catheter hub are no longer in fluid communication with one another. The present inventive injection molded balloon guide catheter hub having a supplemental structure along a section of an inner wall of its through lumen is created by inserting each part of a 3-component hub mold core pin 200 (FIG. 2A) into respective cavities of the hub mold 900 (one half of which is depicted in FIG. 9) prior to injection of the flowable material therein.

Referring to FIGS. 2A-2D, the 3-component hub mold core pin 200, for example, milled or molded, will now be described in detail. An exploded perspective view of the 3-component hub mold core pin 200 is shown in FIG. 2A including: (i) a first component 200a; (ii) a second component 200b; and (iii) a third component 200c. Addressing each component separately, first component 200a of the hub mold core pin 200 includes a proximal cylindrical section 225 having a circular radial cross-section and a distal elliptical section 230 having an elliptical radial cross-section. The outer diameter of the distal elliptical section 230 is larger relative to that of the outer diameter of the proximal cylindrical section 225 forming a single stepped transition in outer diameter 223 therebetween. A central axis of the distal elliptical section 230 is vertically offset from a central axis of the proximal cylindrical section 225, with the two sections “level” or flush at the bottom and all the difference/offset in diameter between the two sections arranged at the top (180° radially relative to the bottom where the two sections 225, 230 are level or flush) forming a stepwise transition 223 in outer diameter. To allow passage of the inflation media from the hub's balloon inflation lumen 130 into the proximal inflation entrance port 335a of the inflation lumen 335 of the catheter shaft 300, the radial depth of the stepwise transition 223 in outer diameter between the distal elliptical section 230 and the proximal cylindrical section 225 of the hub mold core pin 200 used during the injection molding process to form the hub is preferably equal to the radial depth of the eccentrically arranged balloon inflation lumen 335 of the catheter shaft 300. However, the hub's balloon inflation lumen 130 is not in fluid communication (i.e., does not extend radially into or intersect) with the through lumen 325 of the catheter shaft 300 when assembled in the through lumen 120 of the injection molded balloon guide catheter hub 100.

Projecting radially outward from and preferably integral with the outer surface of the first component 200a (including both the proximal cylindrical section 225 and the distal elliptical section 230) of the hub mold core pin 200 are a plurality of fins 205. In a longitudinal/axial direction each fin 205 starts from the distal end/tip of the distal elliptical section 230 and extends into the proximal cylindrical section 225, but stops short of the proximal end. Fins 205 are arranged anywhere along the outer surface where sections 230, 225 are level (i.e., not radially offset) with one another (i.e., along the sides and/or bottom) so as not to interfere with the stepwise transition 223 in outer diameter at which sections 225, 230 are radially offset from one another (i.e., at the top position). Alignment of the distal ends/tips of the fins 205 with that of the distal end/tip of the first component 200a of the hub mold core pin 200 assists in release during withdraw from the hub mold 900 in the opposite direction to the direction of insertion in which it was inserted, as illustrated in FIG. 2D. Proximal ends/tips of the fins 205 terminate distally a predetermined distance (i.e., stops short) of the proximal end/tip of the first component 200a (the exact distance is not critical). As a result, during assembly of the catheter shaft 300 in a distal section 120b of the through lumen 120 of the injection molded balloon guide catheter hub 100, adhesive is prevented from travelling unwantedly into the formed proximal section 120a of the through lumen 120 of the injection molded balloon guide catheter hub 100 formed by the second component 200b of the hub mold core pin 200, potentially blocking/obstructing the entrance of the through lumen 325 of the catheter shaft 300. The first component 200a of the hub mold core pin 200 is placed in the hub mold 900 so that the fins 205 span in a longitudinal/axial direction the region of convergence “S” in which the second internal cavity 910 and the first internal cavity 905 converge with one another. The outer diameter or height in a radial direction of the fins 205 is uniform along its entire longitudinal length.

Starting from a proximal end 210, the second component 200b of the hub mold core pin 200 includes a proximal cylindrical section 208 followed thereafter by a distal conical section 215 tapered smaller towards its distal end which butts up in direct physical contact with the proximal end of proximal cylindrical section 225 of the first component 200a. When assembled in the hub mold 900 the proximal cylindrical section 208 of the second component 200b is aligned with the threads of the through lumen luer 920 of the first internal cavity 905 such that the second component 200b forms the proximal section 120a of the through lumen 120 of the injection molded balloon guide catheter hub 100.

FIG. 2B is a view from the distal end 220 of the 3-component hub mold core pin 200 of FIG. 2A (with the 3-components 200a, 200b, 200c assembled together). By way of illustrative example, the hub mold core pin 200 has four fins 205 projecting radially outward while extending in a longitudinal direction along at least a portion of the distal elliptical section 230 and a portion of the proximal cylindrical section 225. In the exemplary embodiment depicted in the distal end view of FIG. 2B and radial cross-sectional view of FIG. 2C along lines II(C)-II(C), adjacent fins 205 are separated radially equidistantly (90°) from one another. That is, each of the four fins 205 are radially arranged about the 360° outer circumference of the first component 200a of the hub mold core pin 200 at 45°, 135°, 225° and 315°, wherein the 0° reference point is at the point of maximum radial offset between sections 225, 235. In FIGS. 2B & 2C, the radial cross-sectional shape of each fin 205 is preferably a rectangle or a square, however any geometric cross-sectional shape fin may be used to form the complementary geometric shape internal longitudinal channels 140 within the through lumen of the formed injection molded balloon guide catheter hub. Design of the hub mold core pin 200 may be modified, as desired, to include two or more fins the arrangement of which may be selected, as desired, radially equidistant from one another or not. When the three components 200a, 200b, 200c comprising the multi-component hub mold core pin 200 are assembled in the hub mold 900, the radial placement of the fins 205 about the outer surface or profile of the first component 200a of the hub mold core pin 200 do not pass directly through or intersect the region of convergence “S” of the two internal cavities 905, 910 of the hub mold.

FIG. 2D depicts each component 200a, 200b, 200c of the multi-component hub mold core pin 200 assembled independently into respective cavities of one half of the hub mold 900. Specifically, the first component 200a of the hub mold core pin 200 is inserted into the first internal cavity 905 of the hub mold via the distal end (as depicted by the directional arrow at the distal end of the first internal cavity 905 in FIG. 2D). Within the first internal cavity 905 of the hub mold 900 the first component 200a is positioned with the stepped transition 233 in outer diameter between the distal elliptical section 230 and proximal cylindrical section 225 aligned or coinciding with the region of convergence “S” with the second internal cavity 910. The second component 200b of the hub mold core pin 200 is inserted into the first internal cavity 905 of the hub mold 900 via the through lumen luer 920 (as depicted by the directional arrows at the proximal end of the first internal cavity 905 in FIG. 2D). When inserted from opposite ends into the first internal cavity 905 of the hub mold 900, first and second components 200a, 200b of the hub mold core pin 200 abutting one another together form the through lumen 120 of the formed injection molded balloon guide catheter hub 100. The third component 200c of the hub mold core pin 200 is insertable in the second internal cavity 910 of the hub mold 900 (as depicted by the directional arrow at the proximal end or entrance to the second internal cavity 910 in FIG. 2D) until butting up in direct physical contact with an outer surface along a proximal portion of the distal elliptical section 230 of the first component 200a. Proximal and distal sections 120a, 120b together comprising the through lumen 120 of the injection molded balloon guide catheter hub are formed by the second and first components 200a, 200b, respectively, while the hub's balloon inflation lumen 130 is formed by the third component 200c.

With the 3-components 200a, 200b, 200c of the hub mold core pin 200 assembled in their respective internal cavities of one half of the hub mold 900 (as shown in FIG. 2D) the other half of the hub mold is aligned on top thereof and the two halves are secured together. A biocompatible meltable material (e.g., polycarbonate) heated to a predetermined melting temperature in a flowable state is then injected into the respective cavities 905, 910 of the two assembled halves of the hub mold 900 secured together. Specifically, the injected material fills: (i) the first internal cavity 905 of the hub mold 900 surrounding the first and second components 200a, 200b of the hub mold core pin 200 disposed therein (forming the respective distal and proximal sections 120b, 120a, respectively, together comprising the through lumen 120 of the formed injection molded balloon guide catheter hub 100); and (ii) the second internal cavity 910 of the hub mold 900 surrounding the third component 200c of the hub mold core pin 200 therein (forming the balloon inflation lumen 130 of the formed injection molded balloon guide catheter hub 100). Before the injected melted material solidifies, a tool (e.g., pin) is pressed radially inward through side holes 935, 935′ defined in the hub mold 900 into the melted material creating adhesive ports 135, 135′ in the hub's through lumen 120 (as shown in FIG. 2E) into which adhesive is later injectable bonding the catheter shaft 300 assembled therein, as described in detail further below. At least one side hole 935 is provided in the hub mold 900 on each side (i.e., proximal and distal) of the region of convergence “S” between the two cavities 905, 910. Following the injection molding process, the hub mold core pin 200 is withdrawn (i.e., independent removal of each of the 3-components 200a, 200b, 200c in an opposite/reverse direction in which that component was inserted/assembled as depicted by the directional arrows in FIG. 2D) from the hub mold 900.

Thereafter, the two halves of the hub mold 900 are opened/separated. What remains is the formed injection molded balloon guide catheter hub 100 having a through lumen 120 (comprising both proximal and distal sections 120a, 120b, respectively) and a balloon inflation lumen 130 converging therewith in a Y-shape, as depicted in FIGS. 2E & 2F. Within the hub's through lumen 120 along an axial/longitudinal direction spanning the region of convergence (where the two lumens 120, 130 converge) internal longitudinal channels 140 are created by the fins 205 of the hub mold core pin 200 during the injection molding process.

Next, the catheter shaft 300 having a through lumen 325 (e.g., main lumen) and an eccentrically arranged balloon inflation lumen 335 (FIG. 1B) is assembled in the through lumen 120 of the present inventive formed injection molded balloon guide catheter hub 100 (FIGS. 2E & 2F). Catheter shaft 300 is positioned with the proximal inflation entrance port 335a of the balloon inflation lumen 335 located just distal of the region of convergence “S” of the two lumen 120, 130 in the fully formed injection molded balloon guide catheter hub 100 (i.e., in FIGS. 1B & 2D the “*” are aligned with one another). Catheter shaft 300 has a smaller outer profile/diameter relative to the maximum inner diameter/profile of the through lumen 120 of the formed injection molded balloon guide catheter hub 100 in which it is received establishing a radial clearance space therebetween into which a biocompatible adhesive material is injectable via the adhesive ports 135, 135′ and when cured, bonds the assembled components together.

Referring to FIG. 3A, the biocompatible adhesive (depicted by the cross-hatch shading) injected via the adhesive ports 135, 135′ fills the radial clearance space between the catheter shaft 300 assembled in the through lumen 120 of the formed injection molded balloon guide catheter hub 100. Along the hub's through lumen 120 at least one adhesive port 135, 135′ is located on each side (i.e., proximal and distal) of the region of convergence “S” between the hub's balloon inflation lumen 130 and hub's through lumen 120. By way of example, the injection molded balloon guide catheter hub 100 in FIG. 2E has four adhesive ports in total, two on each side (i.e., proximal and distal side) of the region of convergence “S.” The number and arrangement of the adhesive ports 135, 135′ along the hub's through lumen 120 may be selected, as desired, so long as none coincide, align, or overlap with the region of convergence “S” where the hub's balloon inflation lumen 130 converges with the hub's through lumen 120. The adhesive injected into the adhesive holes 135, 135′ fills the radial clearance space defined between the catheter shaft 300 assembled in the through lumen 120 of the injection molded balloon guide catheter hub 100 bonding the components together. Specifically, the adhesive material injected into the adhesive holes 135, 135′ creates a proximal radial bond 305 and a distal radial bond 307, respectively, one on each side of the region of convergence “S” where the through lumen and balloon inflation lumen 120, 130, respectively, of the injection molded balloon guide catheter hub 100 converge with each other (FIG. 3A). Furthermore, each radial bond 305, 307 fills radially 360° the clearance space 355 between the assembled components (FIG. 3D). In addition to forming the proximal and distal radial bonds 305, 307, the injected biocompatible adhesive seeps, flows, travels therebetween through the internal longitudinal channels 140 formed along the inner wall of the through lumen 120 of the injection molded balloon guide catheter hub 100 spanning in a longitudinal direction the region of convergence “S.” The biocompatible adhesive filling the internal longitudinal channels 140 create internal intermediate longitudinal reinforcing bond ribs 315 that span in an axial/longitudinal direction the region of convergence “S.” However, the internal intermediate longitudinal reinforcing bond ribs 315 are radially arranged so as not to obstruct, interfere, block, or limit the flow of inflation media passing from the hub's balloon inflation lumen 130 into the proximal inflation entrance port 335a of the balloon inflation lumen 335 of the catheter shaft 300 assembled therein. This is depicted in FIG. 3A wherein the area directly below/beneath the region of convergence “S” where the two lumens 120, 130 of the hub converge is free or devoid from cross-hatch shading (depicting an area devoid of adhesive).

FIGS. 3B & 3C represent radial cross-sectional views of the through lumen 120 within the injection molded balloon guide catheter hub 100 through the distal radial bond 307 and proximal radial bond 305, respectively. Distally of the region of convergence “S” where the two lumens 120, 130 converge, the eccentric arrangement of the balloon inflation lumen 335 and through lumen 325 of the catheter shaft 300 is shown in FIG. 3B. Between the two radial bonds 305, 307 the injected adhesive flows (i.e., naturally wicks) through the plurality of longitudinal channels 140 (created during the injection molding process of forming the balloon guide catheter hub 100 by the fins 205 of the hub mold core pin 200 placed in the first internal cavity 905 of the hub mold 900) forming internal intermediate longitudinal reinforcing bond ribs 315 extending between the respective radial bonds 305, 307. These internal intermediate longitudinal reinforcing bond ribs 315 span the region of convergence “S” between the hub's through lumen 120 and the balloon inflation lumen 130 (i.e., the proximal inflation entrance port 335a of the catheter shaft 300 being aligned with the region of convergence “S”). FIG. 3D is a representative radial cross-sectional view of the assembly through the region of convergence “S” where the two shaft lumens 120, 130 of the injection molded balloon guide catheter hub 100 converge, i.e., through the internal intermediate longitudinal reinforcing bond ribs 315. As is evident in FIG. 3D, along each of the plurality of longitudinal channels 140 filed with the injected adhesive the plurality of internal intermediate longitudinal reinforcing bond ribs 315 are created in the radial clearance space 355 between the two assembled components (e.g., catheter shaft 300 and through lumen 120 of the injection molded balloon guide catheter hub 100). None of the plurality of internal intermediate longitudinal reinforcing bond ribs 315 obstruct, interfere, limit, block, or restrict passage of the inflation media from the balloon inflation lumen 130 of the injection molded balloon guide catheter hub 100 into the proximal inflation entrance port 335a of the balloon inflation lumen 335 of the catheter shaft 300. Between the proximal and distal radial bonds 305, 307, that portion of the radial clearance space 355 not proximate one of the internal intermediate longitudinal channels 140 filled with adhesive remains open, free, and clear of adhesive (radial clearance space 355 free of cross-hatch shading in FIG. 3D). In the radial cross-sectional view of FIG. 3D it is clearly visible that the internal intermediate longitudinal reinforcing bond ribs 315 are arranged radially (i.e., separated by a predetermined arc length “r”) sufficient to ensure that none of the internal intermedial longitudinal reinforcing bond ribs 315 created by the injected adhesive therein obstructs, interferes, restricts, or blocks in any way the inflation media passing from the hub's balloon inflation lumen 130 through the region of convergence “S” and entering the proximal inflation entrance port 335a of the balloon inflation lumen 335 of the catheter shaft 300. As a result of the internal intermediate longitudinal reinforcing bond ribs 315, that portion of the through lumen 335 of the catheter shaft 300 spanning the region of convergence “S” in the injection molded balloon guide catheter hub 100 is designed to withstand spikes or peaks in pressure during aggressive inflation of the balloon without risk of collapse or narrowing.

To summarize, the present inventive balloon guide catheter hub assembly begins with the insertion independently of each of the components 200a, 200b, 200c of the multi-component hub mold core pin 200 into associated internal cavities 905, 910 of one half of the hub mold 900. With the multi-component hub mold core pin 200 disposed therein the two halves of the hub mold 900 are aligned with one another and secured together. A heated thermoplastic or other reflowable material is injected into the respective internal cavities 905, 910 of the two halves of the hub mold 900 secured together surrounding each of the components 200a, 200b, 200c of the hub mold core pin 200 disposed therein. Before the injected material has cooled/hardened, the adhesive ports 135, 135′ are created by insertion of a pin or other tool via the side holes 935, 935′ defined in the side of each half of the hub mold 900 into the first internal cavity 905 on each side (e.g., proximal and distal sides) of the region of convergence between the two cavities 905, 910.

Once the injected material has cooled/solidified/hardened, each of the 3-components 200a, 200b, 200c forming the hub mold core pin 200 are independently removed (i.e., withdrawn in a reverse direction to that during insertion into their associated internal cavities of the hub mold). Next, both halves of the hub mold 900 are opened/separated leaving the formed injection molded balloon guide catheter hub 100 with the plurality of longitudinal channels 140 (created during the injection molding process by the fins 205 of the first component 200a of the hub mold core pin 200) disposed along the through lumen 120 in a longitudinal section spanning the region of convergence “S” of the lumens 120, 130. The proximal end of the multi-lumen catheter shaft 300 is inserted into the distal end of the distal section 120b of the through lumen 120 of the formed injection molded balloon guide catheter hub 100. Through the adhesive ports 135, 135′ the biocompatible adhesive is injected into the clearance space 355 defined between the inner wall of the distal section 120b of the through lumen 120 of the formed injection molded balloon guide catheter 100 and the outer surface of the catheter shaft 300 assembled therein securing the components together. Specifically, the injected biocompatible adhesive forms 360° radial bonds 305, 307 on each side/face (e.g., proximal radial bond and distal radial bond) of the region of convergence “S” of the two lumens 120, 130 of the injection molded balloon guide catheter hub 100. Between the respective radial bonds (e.g., proximal and distal radial bonds) 305, 307, the injected adhesive travels (i.e., seeps) along the axial/longitudinal channels 140 of the distal section 120b of the through lumen 120 of the formed injection molded balloon guide catheter hub 100 producing internal intermediate longitudinal reinforcing bond ribs 315. The through lumen 325 of the catheter shaft when assembled in the through lumen of the injection molded balloon guide catheter hub is able to withstand pressure spikes or peaks during aggressive inflation of the balloon without collapsing or narrowing as a result of the reinforcement provided by these internal intermediate longitudinal reinforcing bond ribs 315. Respective proximal and distal radial bonds 305, 307 prevent inflation media exiting from the hub's balloon inflation lumen 130 from entering the through lumen 325 of the catheter shaft 300, instead directing the pressurized inflation media to travel only in a distal direction into the proximal inflation entrance port 335a of the balloon inflation lumen 335 of the catheter shaft 300. By way of illustrative example, the internal intermediate longitudinal reinforcing bond ribs 315 are approximately 3 mm in length in a longitudinal/axial direction. The injection molded balloon guide catheter hub 100 is preferably provided with a strain relief 150 positioned about the protruding lug 915 to prevent kinking of the catheter shaft 300 assembled therein.

An alternative configuration for preventing possible collapse or narrowing of the through lumen of the catheter shaft during aggressive inflation of the balloon is to introduce a mechanical flow restrictor internally in the balloon inflation lumen of the injection molded balloon guide catheter hub preventing the occurrence of peak/spikes in pressure on the distal side of the flow restrictor.

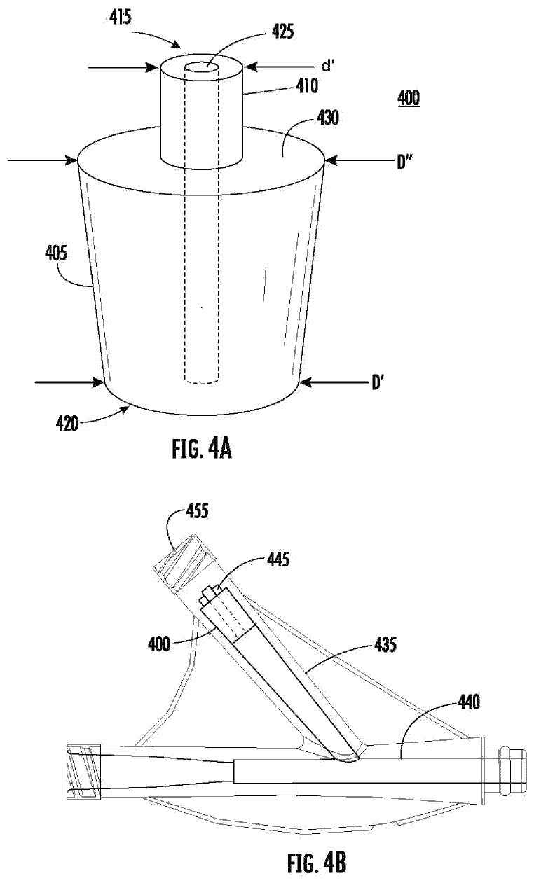

FIG. 4A is a perspective view of the present inventive flow restrictor 400 having a proximal end 415 and an opposite distal end 420. Starting at the proximal end 415 the flow restrictor 400 comprises a chimney section 410 followed thereafter by a tapered/conical main section 405 terminating in the distal end 420. A passageway or channel 425 extends axially/longitudinally through the flow restrictor 400 from its proximal end 415 to its opposite distal end 420. Typically, the inner diameter of the balloon inflation lumen 435 of the injection molded balloon guide catheter hub 400 is tapered so in such circumstances the shape of the main section 405 of the flow restrictor 400 is also tapered/conical to conform therewith. The outer diameter of the tapered main section 405 is sized to friction/interference fit within the hub's balloon inflation lumen 435. Chimney section 410 has an outer diameter (d′) smaller than the outer diameter (D″) at the proximal end of the main section 405 with the radial offset therebetween forming a stepped transition or shoulder 430. In addition, the outer diameter (d′) of the chimney section 410 is smaller than that of the outer diameter (D′) at the distal end of the main section 405 which, in turn, is smaller than the outer diameter (D″) at the proximal end of the main section 405. It is also noted that the outer diameter “d” of the chimney section 410 depicted in FIGS. 4A & 4B is cylindrical and uniform, but need not be, so long at the interface the diameter of the chimney section 410 is smaller relative to that of the main section 405 forming the stepped transition or shoulder 430.

During assembly, the distal end 420 of the present inventive flow restrictor 400 is introduced internally into the balloon inflation lumen 435 within the injection molded balloon guide catheter hub via the balloon inflation lumen luer 455. As previously mentioned, the tapered/conical main section 405 is sized and shaped to conform with that of the tapered balloon inflation lumen 435 within the injection molded balloon guide catheter hub forming a friction/interference fit therebetween. Positioning or placement of the flow restrictor 400 in the balloon inflation lumen 435 within the injection molded balloon guide catheter hub is such that the distal end 420 of the flow restrictor does not extend into the region of convergence of the hub's balloon inflation lumen 435 and through lumen 440, as shown in FIG. 4B. Similarly, the proximal end 415 of the flow restrictor 400 does not interfere with a syringe luer tip (not shown) when connected to the balloon inflation lumen luer 455 of the injection molded balloon guide catheter hub. With the flow restrictor 400 lodged via a friction/interference fit in the balloon inflation lumen 435 of the injection molded balloon guide catheter hub the area bound distally by the shoulder 430, the outer wall of the chimney section 410, and the inner wall of the hub's balloon inflation lumen 435 are filled with adhesive 445 permanently securing the flow restrictor 400 in place to prevent dislodgement or movement (being sucked out in a proximal direction) during deflation of the balloon. Adhesive is prohibited from flowing into the channel 425 or distally beyond the shoulder 430 of the flow restrictor.

During aggressive inflation of the balloon, inflation media injected by a syringe connected to the balloon inflation lumen luer 455 flows through the channel 425 of the flow restrictor 400 permanently secured (adhered) in the balloon inflation lumen 435 within the injection molded balloon guide catheter hub. Inflation media flows through the reduced inner diameter of the channel 425 disposed in the hub's balloon inflation lumen 435 at a reduced rate (relative to the flow rate through the inflation lumen without the flow restrictor) avoiding peaks/spikes in pressure in the injection molded balloon guide catheter hub on the distal side of the flow restrictor thereby preventing collapse or narrowing of the through lumen of the catheter shaft.

The two aforementioned novel designs target different aspects of the injection molded balloon guide catheter hub but achieve the same goal of preventing collapse or narrowing of the through lumen of the catheter shaft during aggressive inflation of the balloon. In the first described novel design set forth in FIGS. 3A-3D the formation of internal intermediate longitudinal reinforcing bond ribs in the clearance space between the catheter shaft and the through lumen of the injection molded balloon guide catheter hub resist collapse or narrowing when subject to peaks/spikes in pressure during aggressive inflation of the balloon. Whereas, the second flow restrictor design in FIGS. 4A & 4B focuses on avoiding pressure peaks/spikes distally thereof in the injection molded balloon guide catheter hub during aggressive inflation of the balloon by restricting the rate of flow of the inflation media through the hub's balloon inflation lumen.

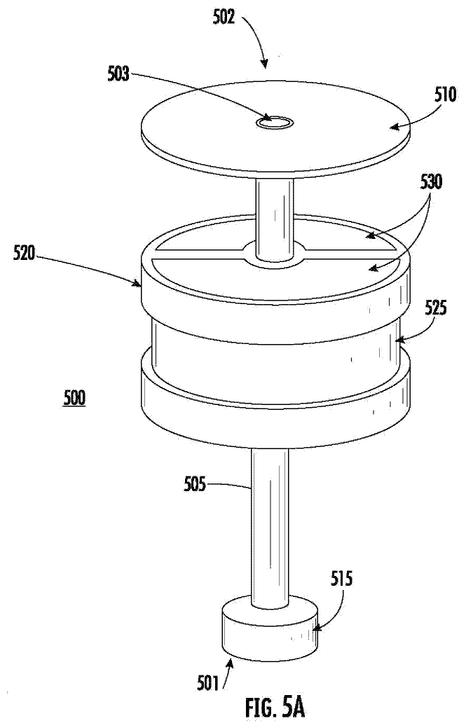

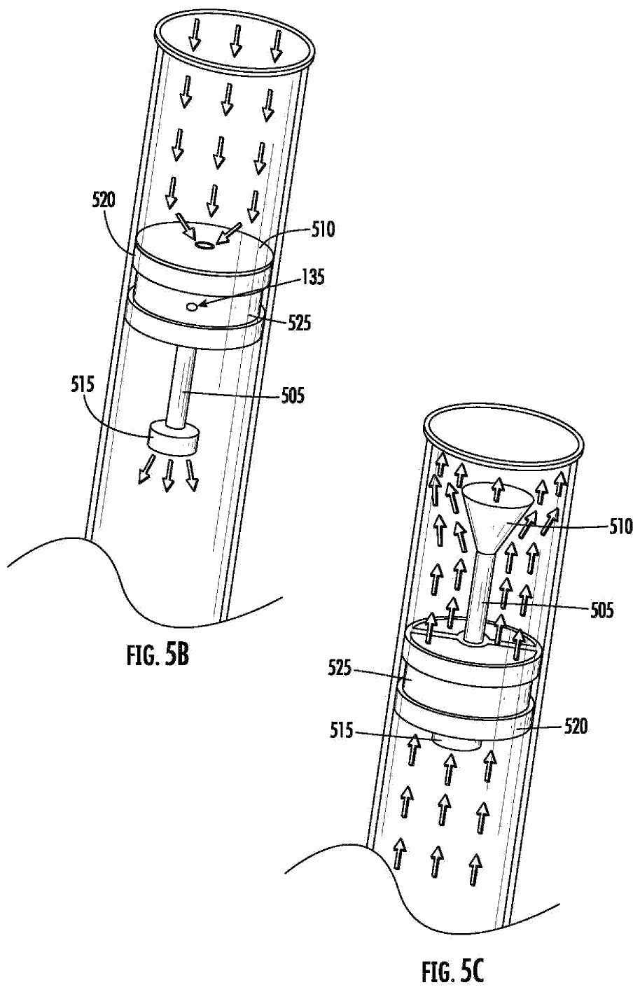

The flow restrictor mechanical device 400 in FIGS. 4A & 4B disadvantageously restricts or limits the flow rate of the inflation media at all times (i.e., during both inflation and deflation of the balloon). By restricting the rate of flow, flow restrictor 400 undesirably negatively impacts time performance during deflation, in contradiction to a desire by interventionalists to minimize balloon deflation time. This disadvantage is overcome by designing a self-transitioning flow restrictor 500 (FIGS. 5A-5C) that automatically (i.e., without manual intervention) transitions between engagement and disengagement. Specifically, the present inventive flow restrictor 500 self-engages under positive pressure (turns ‘ON’ to limit or restrict the fluid flow rate of the inflation media) during balloon inflation and self-disengages under negative pressure (turns ‘OFF’ to allow an increased or greater rate of flow of the inflation media relative to the limited or restricted flow rate during the self-engaged (ON′) state) during balloon deflation. During inflation, the flow restrictor self-engages to restrict the rate of flow of inflation media during inflation of the balloon thereby preventing or minimizing pressure spikes/peaks distally thereof in the injection molded balloon guide catheter hub. However, the flow restrictor automatically self-disengages (allowing an increased or greater rate of flow of inflation media therethrough) during deflation of the balloon so as to minimize any negative impact on deflation time performance.

Referring to FIG. 5A, the self-transitioning flow restrictor 500 includes a restrictor needle 505 (e.g., a tube with a longitudinal passageway 503) having a distal end 501 and an opposite proximal end 502. Longitudinal passageway 503 has an inner diameter preferably in a range of approximately 0.2 mm-approximately 0.5 mm. A flexible seal 510 (e.g., rubber disc) is fixedly/securely mounted about the proximal end 502 of the restrictor needle 505. At the opposite distal end of the restrictor needle 505 a stopper 515 (e.g., rubber disc) is fixedly/securely mounted thereto. Longitudinal passageway 503 remains open at both ends (i.e., the passageway 503 is neither closed off nor obstructed by the flexible seal 510 or the stopper 515 mounted about each end of the restrictor needle 505) to allow the free flow of inflation media therethrough regardless of the state of the flow restrictor. A cylindrical body 520 is radially disposed about the restrictor needle 505 between the flexible seal 510 and the stopper 515. Restrictor needle 503 is axially slidable through a central hole formed in cylindrical body 520. The cylindrical body 520 has a tapered outer cylindrical contour corresponding to the internal geometry of the balloon inflation lumen within the injection molded balloon guide catheter hub. One or more interior cavities 530 are defined radially inward of the outer surface of the cylindrical body 520 through which the inflation media during deflation of the balloon flows therethrough when the flow restrictor is in an ‘OFF’ state (e.g., deactivated or disengaged). Two interior cavities are shown in the example of FIGS. 5A-5C, but any number of one or more interior cavities are possible. The radial outer wall of the cylindrical body 520 has a radial recess 525 fillable with an injectable adhesive after being assembled in the balloon inflation lumen within the injection molded balloon guide catheter hub via the balloon inflation lumen luer to prevent dislodgement and/or leakage during use. The outer diameter of the stopper 515 is smaller relative to the outer diameter of the cylindrical body 520 so that during deflation of the balloon when the flow restrictor is in the disengaged (“OFF”) state the inflation media is able to pass through the internal cavities 530 of the cylindrical body 520.

During assembly, the flow restrictor 500 is introduced into the balloon inflation lumen within the injection molded balloon guide catheter hub until the recess 525 is aligned with an adhesive port defined radially inward through a side of the balloon inflation lumen of the injection molded balloon guide catheter hub. An adhesive is injected through the adhesive port of the injection molded balloon guide catheter hub filling the recess 525 preventing dislodgement of and leakage around the cylindrical body 520. In use (i.e., during inflation/deflation of the balloon) the cylindrical body 520 of the flow restrictor 500 is held in position within the balloon inflation lumen of the injection molded balloon guide catheter hub while the flow restrictor automatically transitions between “ON”/“OFF” (e.g., engaged/disengaged) states by axially sliding the restrictor needle 503 in a proximal/distal direction through the central hole formed in the body 520 in response to positive or negative pressure experience during inflation or deflation of the balloon respectively.

FIG. 5B depicts the flow restrictor of FIG. 5A in an ‘ON’ (i.e., engaged or activated) state while inflation media is introduced via a syringe connected to the balloon inflation luer of the injection molded balloon guide catheter hub to inflate the balloon. Arrows depict the flow of inflation media through the flow restrictor 500 during inflation of the balloon, wherein the force of the inflation media on the flexible seal 510 while flowing through the balloon inflation lumen of the injection molded balloon guide catheter hub causes the restrictor needle 503 to slide in a distal direction relative to that of the body 520 (which remains secured in position within the hub's balloon inflation lumen via the adhesive) until the flexible seal 510 is seated in direct physical contact with proximal end of the cylindrical body 520. In this “seated” position, the planar/flat flexible seal 510 blocks or prohibits the flow of the inflation media through the interior cavities 530 of the body 520, the rate of flow being restricted or limited exclusively through the narrow passageway 503 (reduced inner diameter) of the restrictor needle 505. The limiting or restricting of the rate of flow of inflation media through the balloon inflation lumen within the injection molded balloon guide catheter hub in such manner minimizes or prevents pressure spikes or peaks distally of the flow restrictor during aggressive inflation of the balloon.

During deflation of the balloon, with the application of negative pressure created by the vacuum syringe, the flow of the inflation media is in the opposite direction (e.g., proximal direction), as depicted by the arrows in FIG. 5C, automatically transitions the flow restrictor 500 to an ‘OFF’ (i.e., disengaged or deactivated) state (without the need for manual hand manipulation). Specifically, under negative pressure the inflation media flows in a proximal direction through the interior cavities 530 of the body 520 imposing a force (in the same direction) on the distal side of the flexible seal 510 lifting it off from (separate a predetermined distance from so no longer in direct physical contact with) the proximal end of the body 520 as the restrictor needle 503 slides/moves relative to the body 520 allowing the inflation media to exit through the proximal end. Movement of the restrictor needle 503 in a proximal direction ceases when the stopper 515 is in direct physical contact with the distal end of the cylindrical body 520. Under negative pressure, the flexible seal 510 deflects/inverts into a conical shape (with its widest end oriented furthest away from the cylindrical body 520) reducing its cross-sectional area allowing the free flow of inflation media around and beyond the flexible seal in a proximal direction. With the flexible seal 510 in a fully elevated position (i.e., position fully separated relative to that of the body 520) exposing the interior cavities and reducing the cross-sectional area of the flexible seal, the inflation media is allowed to flow at an increased rate through the interior cavities of the body and around the inverted flexible seal having minimal, if any, effect on deflation time performance. Thus, transitioning of the flow restrictor 500 between “ON”/“OFF” states occurs solely as a result of the force imposed by the inflation media on the flexible seal 510 during inflation/deflation, respectively, of the balloon without the need for manual hand manipulation of the flow restrictor or use of a mechanical spring device.

Instead of restricting the rate of flow of inflation media using a flow restrictor as described above with respect to FIGS. 4A, 4B, 5A-5C, in yet another alternative configuration peaks or spikes in pressure in the balloon inflation lumen of the injection molded balloon guide catheter hub during aggressive inflation of the balloon may be counterbalanced (i.e., compensated for) by increasing the internal volume within the hub's balloon inflation lumen. This may be achieved by using a mechanical volume expanding element such as an elastomeric element (e.g., sleeve, patch, or tube) associated with the balloon inflation lumen of the injection molded balloon guide catheter hub to increase the internal volume therein, temporarily during aggressive inflation of the balloon.

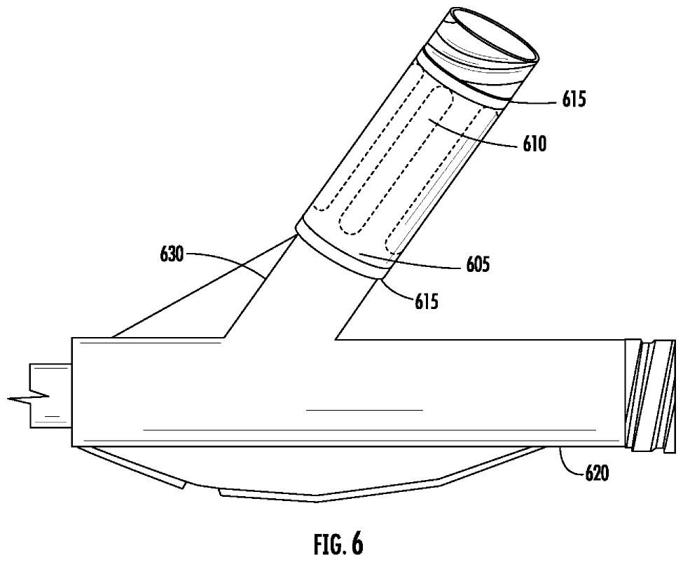

In the example shown in FIG. 6, an elastomeric sleeve 605 (e.g., a semi-compliant material such as silicone) covers or encloses a plurality of axial/longitudinal slits 610 defined about the circumference of the hub's balloon inflation lumen 630. Four slits 610 are shown all identical in shape length, width, and spacing between, however, any number of one or more slits are possible and within the scope of the invention the shape, size, and spacing of each may be selected, as desired. Elastomeric sleeve 605 is sealed along its proximal and distal ends/edges about the outer surface of the balloon inflation lumen 630 of the injection molded balloon guide catheter hub via a ring of adhesive 615. If desired, adhesive may also be placed on the outer surface of the hub's balloon inflation lumen between adjacent slits. During aggressive inflation, the pressure of the inflation media in the balloon inflation lumen 630 of the balloon guide catheter hub increases. Upon exceeding a predetermined pressure threshold, such heightened pressure is counterbalanced by the elastomeric sleeve 605 expanding radially outward (i.e., swells), increasing the internal volume in the hub's balloon inflation lumen 630. An added benefit provided by the elastomeric sleeve is that it serves as a grip for the interventionalist while assembling accessories to the balloon guide catheter hub and handling the device during the procedure.

Rather than being a single sleeve as represented in FIG. 6, the elastomeric material may be a plurality of elastomeric coverings (e.g., patches) secured to the outer surface of the balloon inflation lumen within injection molded balloon guide catheter hub, each covering (e.g., patch) enclosing one or more openings defined in the outer wall of the hub's balloon inflation lumen 730.

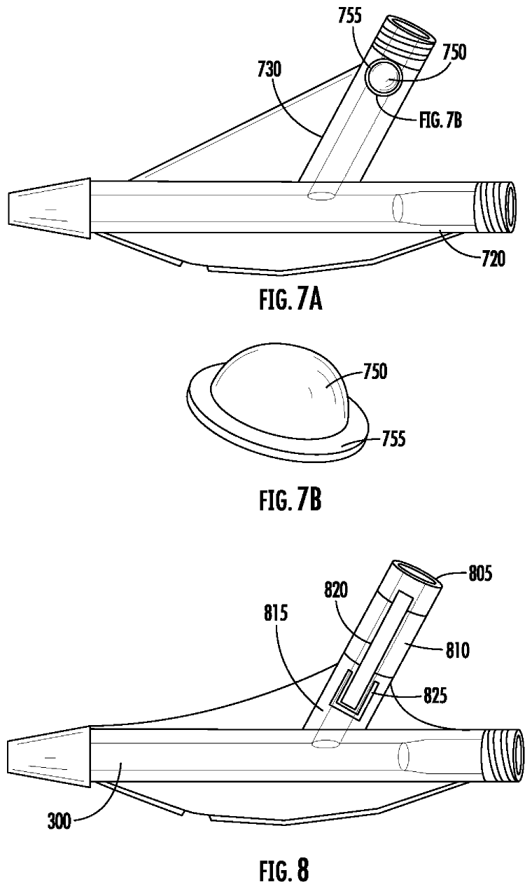

Such plurality of elastomeric coverings may be circular in shape forming a blister-like expansion, that is, a flexible elastomeric covering that expands or bulges under pressure from the inflation media during aggressive inflation of the balloon and out from the opening(s) beneath the covering. By way of example, FIGS. 7A & 7B depict a single blister pressure limiter. An orifice or opening is defined in the side wall of the balloon inflation lumen 730 within the injection molded balloon guide catheter hub. Positioned over the orifice or opening is a circular patch of an elastomeric material forming a blister 750 that is hermetically sealed against the outer surface of the balloon inflation lumen 730 of the balloon guide catheter hub by a flange 755. The elastomeric covering forming the blister swells when the pressure in the hub's balloon inflation lumen 730 exceeds a predetermined pressure threshold. Inflation media is injected (e.g., via a syringe connected to the balloon inflation lumen luer) into the balloon inflation lumen 730 of the injection molded balloon guide catheter hub to inflate the balloon. During aggressive inflation, any spike or peak in pressure of the inflation media that exceeds the predetermined pressure threshold causes the blister 750 to swell/expand radially outward increasing the overall internal volume thereby avoiding, preventing or limiting the increase in internal pressure (i.e., peaks or spikes in pressure) in the hub's balloon inflation lumen.

The openings in the hub's balloon inflation lumen 730 may be any geometric shape (e.g., circular). One or more elastomeric covering(s) preferably, but need not necessarily, conform in shape (e.g., circular) with that of the corresponding opening. Each elastomeric patch or covering may be associated with and cover only a single opening defined in the balloon inflation lumen within the injection molded balloon guide catheter hub on a one-to-one ratio. It is also possible that each or some of the plural elastomeric coverings may be associated with so as to cover more than one (but less than all) of the openings defined in the outer surface of the balloon inflation lumen within the injection molded balloon guide catheter hub. Furthermore, each elastomeric covering is secured (e.g., adhesively and/or mechanically) to the outer surface of the balloon inflation lumen of the injection molded balloon guide catheter hub around the one or more openings to be covered.

Instead of using an adhesive ring to secure the elastomeric sleeve or covering to the outer surface of the balloon inflation lumen of the injection molded balloon guide catheter hub, as illustrated in FIG. 6A, a mechanical restraining device may be employed to ensure that an elastomeric tube comprising one section of a multi-component balloon inflation lumen of the injection molded balloon guide catheter hub is properly aligned and secured together. In this alternative embodiment represented in FIG. 8 the flow restrictor comprises one of three separate, independent, and distinct components that when assembled together form the balloon inflation lumen of the injection molded balloon guide catheter hub (depicted with the catheter shaft inserted in the through lumen of the injection molded balloon guide catheter hub). Specifically, the hub's balloon inflation lumen 820 includes an elastomeric tube component 810 (e.g., silicone tube) disposed between a proximal section balloon inflation lumen component 805 and a distal section balloon inflation lumen component 815.

Elastomeric tube component 810, once aligned both longitudinally and radially with the other two components 805, 815, is maintained secured together and in proper alignment via a purely mechanical system (e.g., a “rail-and-guide” mechanism). In the side view of FIG. 8, a first rail-and-guide mechanism is visible, but a second rail-and-guide mechanism identical to that of the first rail-and-guide mechanism is disposed radially 180° relative to the first (but not visible in FIG. 8). Each rail-and-guide mechanism is identical in construction and includes a rail or arm 820 fixedly secured at one end while its opposite free end is engageable within a constraining element 825 (e.g., U-shape guide or recess). By way of example, a proximal end of the rail or arm 820 is secured to the outer surface of the proximal section balloon inflation lumen component 805, while the constraining element 825 is disposed on the outer surface of the distal section of the balloon inflation lumen component 815 forming part of the body of the hub's balloon inflation lumen. Thus, when the three components 805, 810, 815 comprising the balloon inflation lumen of the injection molded balloon guide catheter hub are properly aligned the free end of the rail 820 is engaged, maintained, or restricted within the constraining element 825 maintaining the three components 805, 810, 815 in proper alignment (i.e., both radially and longitudinally) and secured together. When a predetermined threshold pressure is exceeded in the hub's balloon inflation lumen the elastomeric tube component 810 expands radially and/or longitudinally increasing the internal volume therein to compensate for or counterbalance any increase in pressure of the inflation media during aggressive inflation of the balloon thereby preventing peaks or spikes in pressure. Prior to inflation of the balloon, rail or arm 820 while in a closed state with the free end thereof engaged in the constraining element 825 is offset radially outward relative to the outer surface of the elastomeric tube component 810 a sufficient distance to accommodate without interfering, restricting, or limiting radial outward expansion (swelling) of the elastomeric tube component 810 during subsequent inflation of the balloon. In other words, when the balloon is in a deflated state, there is no direct physical contact between the rail 810 and the outer surface of the elastomeric tube component 810.

The present invention illustrates and describes numerous mechanical solutions to prevent collapse or narrowing of the through lumen of the catheter shaft during aggressive inflation of the balloon. Generically, the different mechanical configurations achieve the same goal in different ways by: a reinforcing structure disposed in the clearance space between the through lumen of the injection molded balloon guide catheter hub and the catheter shaft assembled therein; mechanically controlling the flow rate of inflation media through the balloon inflation lumen within the injection molded balloon guide catheter hub, to prevent pressure spikes; or mechanically increasing the internal volume within balloon inflation lumen of the injection molded balloon guide catheter hub to compensate or counterbalance for pressure spikes. It is noted that any combination of these present inventive configurations may be used independently of one another or simultaneously in any combination thereof in the same injection molded balloon guide catheter hub.

Thus, while there have been shown, described, and pointed out fundamental novel features of the invention as applied to a preferred embodiment thereof, it will be understood that various omissions, substitutions, and changes in the form and details of the systems/devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

What is claimed is:

1. A balloon guide catheter comprising:
an injection molded balloon guide catheter hub including a through lumen and a balloon inflation lumen converging therewith defining a region of convergence; and
a preventative structure in the injection molded balloon guide catheter hub, the preventative structure withstanding, avoiding or counterbalancing pressure peaks in the injection molded balloon guide catheter hub.

2. The balloon guide catheter in accordance with claim 1, wherein the injection molded balloon guide catheter hub has a plurality of longitudinal channels formed along an inner wall of the through lumen spanning the region of convergence; and the balloon guide catheter further comprises a catheter shaft having a through lumen extending from a proximal end to an opposite distal end, an eccentrically arranged balloon inflation lumen and a balloon secured about the catheter shaft; wherein the proximal end of the catheter shaft is assembled in the through lumen of the injection molded balloon guide catheter hub via an adhesive bond therebetween; wherein the adhesive bond includes a proximal 360° radial bond disposed on a proximal side of the region of convergence and a distal 360° radial bond disposed on a distal side of the region of convergence; and wherein the preventative structure comprises a plurality of internal intermediate longitudinal reinforcing bond ribs formed within the plurality of longitudinal channels spanning between the proximal and distal 360° radial bonds formed along the inner wall of the through lumen of the injection molded balloon guide catheter hub spanning the region of convergence where the balloon inflation lumen and the through lumen in the injection molded balloon guide catheter hub converge with one another.

3. The balloon guide catheter in accordance with claim 2, wherein the plurality of internal intermediate longitudinal reinforcing bond ribs are arranged radially so as not to interfere with the region of convergence where the balloon inflation lumen and the through lumen of the injection molded balloon guide catheter hub converge with one another.

4. The balloon guide catheter in accordance with claim 3, wherein the through lumen of the injection molded balloon guide catheter hub has four internal intermediate longitudinal reinforcing bond ribs arranged radially equidistantly from one another.

5. The balloon guide catheter in accordance with claim 1, wherein the preventative structure comprises a flow restrictor disposed in the balloon inflation lumen of the injection molded balloon guide catheter hub; the flow restrictor including a chimney section disposed at a proximal end and a tapered main section disposed at an opposite distal end with a channel extending longitudinally therethrough both sections; the chimney section having an outer diameter smaller than that of an outer diameter at a proximal end of the tapered main section with a radial offset therebetween forming a shoulder; and the tapered main section having an outer diameter at a distal end smaller than the outer diameter at the proximal end of the tapered main section.

6. The balloon guide catheter in accordance with claim 1, wherein the preventative structure comprises a self-transitioning flow restrictor disposed in the balloon inflation lumen of the injection molded balloon guide catheter hub; the flow restrictor comprising:
a restrictor needle having a proximal end and an opposite distal end and a passageway extending therethrough;
a flexible seal mounted about the proximal end of the restrictor needle;
a stopper securely mounted about the distal end of the restrictor needle; the passageway of the restrictor needle remaining open with neither the proximal end nor the distal end closed off by the flexible seal or the stopper; and
a body radially disposed about the restrictor needle and located in an axial direction between the flexible seal and the stopper; the body having a proximal end, an opposite distal end, and at least one interior cavity defined therethrough; the restrictor needle being slidable in a longitudinal direction through the body;
wherein the flow restrictor is transitionable between two states including: (i) a disengaged state in which the stopper is seated on distal end of the body, the flexible seal is separated relative to the proximal end of the body exposing the at least one interior cavity, and the flexible seal is inverted having a minimum outer diameter; and (ii) an engaged state in which the stopper is separated relative to the distal end of the body, the flexible seal is seated on the proximal end of the body closing off the at least one interior cavity, and the flexible seal is planar having a maximum outer diameter.

7. The balloon guide catheter in accordance with claim 1, wherein the balloon inflation lumen of the injection molded balloon guide catheter hub along an outer wall thereof has at least one opening defined radially therein and the preventative structure comprises a flow restrictor made of an elastomeric material secured to an outer surface of the balloon inflation lumen of the injection molded balloon guide catheter hub covering the at least one opening.

8. The balloon guide catheter in accordance with claim 1, wherein the balloon inflation lumen of the injection molded balloon guide catheter hub comprises three components separable from one another including: (i) a first injection molded component, (ii) an elastomeric tube component, and (iii) a third injection molded component; wherein the elastomeric tube component is disposed between the first and third injection molded components; and wherein all three components are axially and radially aligned; the assembly further comprises a releasable mechanical assembly securing the three components together when properly aligned both axially and radially; wherein the releasable mechanical assembly does not physically contact the elastomeric tube component.

9. The balloon guide catheter in accordance with claim 8, wherein the releasable mechanical assembly is a rail-and-guide system.

10. A method of manufacture of a balloon guide catheter, the method comprising the steps of:
assembling two halves of a balloon guide catheter hub mold aligned and secured together to define therein a first internal cavity for forming a through lumen of an injection molded balloon guide catheter hub and a second internal cavity for forming a balloon inflation lumen within the injection molded balloon guide catheter hub; the first internal cavity converging with the second internal cavity along a region of convergence;
introducing into the assembled two halves of the balloon guide catheter hub mold a multi-component hub mold core pin comprising three components including a first component, a second component, and a third component; the first component having a plurality of fins extending in a longitudinal direction and radially outward from an outer surface thereof; each of the first and second components being inserted into the first internal cavity of the assembled two halves of the balloon guide catheter hub mold in proximal and distal directions, respectively; the third component being inserted in a distal direction into the second internal cavity of the assembled two halves of the balloon guide catheter hub mold;
injecting into the assembled two halves of the balloon guide catheter mold a flowable material filling: (i) the first internal cavity surrounding the first and second components of the multi-component hub mold core pin disposed therein forming the through lumen of the injection molded balloon guide catheter hub; and (ii) the second internal cavity surrounding the third component of the hub mold core pin disposed therein forming the balloon inflation lumen of the injection molded balloon guide catheter hub;
pressing a tool radially inward through the injected flowable material in the first internal cavity forming the through lumen of the injection molded balloon guide catheter hub to define a plurality of adhesive ports therein, at least one adhesive port formed on each of a proximal side and a distal side of the region of convergence;
cooling of the injected flowable material in the balloon guide catheter hub mold;
withdraw of each of three components of the multi-component hub mold core pin from the assembled two halves of the balloon guide catheter hub mold; and
opening the assembled two halves of the balloon guide catheter hub mold, revealing the injection molded balloon guide catheter hub having along an axial section of the through lumen spanning the region of convergence in which the balloon inflation lumen and the through lumen of the injection molded balloon guide catheter hub converge a plurality of longitudinal channels formed by the plurality of fins of the first component of the multi-component hub mold core pin during the injection of the flowable material into the assembled two halves of the balloon guide catheter hub mold.

11. The method in accordance with claim 10, wherein the plurality of fins are four fins arranged radially equidistantly.

12. The method in accordance with claim 10, wherein:
the first component of the multi-component hub mold core pin includes a proximal cylindrical section having a circular radial cross-section and a distal elliptical section having an elliptical radial cross-section; the plurality of fins extending at least partially over each of the proximal cylindrical section and the distal elliptical section; wherein the first component of the multi-component hub mold core pin forms a distal section of the through lumen of the injection molded balloon guide catheter hub;
the second component of the multi-component hub mold core pin includes a cylindrical proximal section and a conical distal section; wherein the second component of the multi-component hub mold core pin forms a proximal section of the though lumen of the injection molded balloon guide catheter hub; and
the third component of the multi-component hub mold core pin includes a cylindrical proximal section and a conical distal section terminating in a distal end; the distal end of the third component conforming in shape to an outer surface of the distal elliptical section of the first component of the multi-component hub mold core pin.

13. The method in accordance with claim 10, further comprising the steps of:
inserting a catheter shaft into the through lumen of the injection molded balloon guide catheter hub; the catheter shaft having a through lumen and an eccentrically arranged balloon inflation lumen;
injecting via the plurality of adhesive ports an adhesive filling a clearance space between the through lumen of the injection molded balloon guide catheter hub and the catheter shaft inserted therein; the injected adhesive forming on each side of the region of convergence a 360° radial proximal bond and a 360° radial distal bond, respectively; and between the 360° radial proximal and distal bonds the injection adhesive filling the plurality of longitudinal channels creating a plurality of internal intermediate longitudinal rib bonds; none of the plurality of internal intermediate longitudinal rib bonds interfering with the region of convergence where the balloon inflation lumen and the through lumen of the injection molded balloon guide catheter hub converge.

14. A method of operating a balloon guide catheter including: a catheter shaft having a through lumen extending from a proximal end to an opposite distal end and an eccentrically arranged balloon inflation lumen; a balloon is secured about a distal section of the outer surface of the catheter shaft; and an injection molded balloon guide catheter hub including a through lumen receiving therein the proximal end of the catheter shaft and a balloon inflation lumen converging therewith defining a region of convergence; the method comprising the steps of;
during aggressive inflation of the balloon, preventing collapse or narrowing of the through lumen of the catheter shaft in the region of convergence via a mechanical preventative structure disposed in the balloon inflation lumen of the injection molded balloon guide catheter hub.

15. The method in accordance with claim 14, wherein the mechanical preventative structure is a flow restrictor limiting a rate of flow of inflation media through the balloon inflation lumen of the injection molded balloon guide catheter hub distally of the mechanical preventative structure.

16. The method in accordance with claim 15, wherein the flow restrictor includes: a restrictor needle having a proximal end and an opposite distal end and a passageway extending therethrough; a flexible seal mounted about the proximal end of the restrictor needle; a stopper securely mounted about the distal end of the restrictor needle; the passageway of the restrictor needle remaining open in that neither the proximal end nor the distal end being closed off by the flexible seal or the stopper; and a body radially disposed about the restrictor needle and located in an axial direction between the flexible seal and the stopper; the body having a proximal end, an opposite distal end, and at least one interior cavity defined therethrough; the restrictor needle being slidable in a longitudinal direction through the body;
self-transitioning of the flow restrictor between two states: (i) an engaged state during inflation wherein the stopper is separated from the distal end of the body while the flexible seal is planar, having a maximum outer diameter, and seated on the proximal end of the body, restricting flow exclusively through the passageway of the restrictor needle; and (ii) a disengaged state during deflation wherein the stopper is seated on the distal end of the body while the flexible seal is separated relative to the proximal end of the body and in an inverted non-planar shape having a minimum outer diameter allowing flow through the interior cavity of the body, around the inverted flexible seal and through the passageway of the restrictor needle.

17. The method in accordance with claim 14, wherein the mechanical preventative structure increases internal volume in the balloon inflation lumen of the injection molded balloon guide catheter hub.

18. The method in accordance with claim 17, wherein the mechanical preventative structure is an elastomeric material secured to an outer surface of the inflation lumen shaft of the balloon guide catheter hub, the elastomeric material expanding radially outward in response to the internal pressure within the balloon inflation lumen of the injection molded balloon guide catheter hub exceeding the predetermined threshold.

19. The method in accordance with claim 17, wherein the mechanical preventative structure is an elastomeric tube comprising part of the balloon inflation lumen of the injection molded balloon guide catheter hub; the parts of the balloon inflation lumen of the injection molded balloon guide catheter hub including the elastomeric tube being secured together via a releasable mechanical assembly; the elastomeric tube expanding radially outward and/or longitudinally in response to the internal pressure within the balloon inflation lumen of the injection molded balloon guide catheter hub exceeding the predetermined threshold without interference from the releasable mechanical assembly.
